(19) United States
(12) Patent Application Publication
(10) Pub. No.: US 2020/0229401 A1
(43) Pub. Date: Jul. 23, 2020

(54) PRODUCTS, SYSTEMS, AND METHODS FOR PREDICTIVE HEALTH AND NUTRITION MODELING AND FULFILLMENT FOR ANIMALS

(71) Applicant: PSK INNOVATIONS, LLC, Miramar, FL (US)

(72) Inventors: STEVE O HERNANDEZ,, MIRAMAR, FL (US); PEDRO MORAS, Fort Lauderdale, FL (US); KASRA FARSAD, SAN JOSE, CA (US)

(21) Appl. No.: 16/646,114

(22) PCT Filed: Dec. 15, 2017
(86) PCT No.: PCT/US17/66846

§ 371 Date: Mar. 10, 2020

Related U.S. Application Data
(60) Provisional application No. 62/436,445, filed on Dec. 20, 2016.

(51) Int. Cl. A01K 29/00; A01K 11/00; A01K 5/01; A01K 5/02; G01G 19/22; G01G 21/22; G16H 50/30; G16H 20/60

(52) U.S. Cl. CPC A01K 29/005; A01K 11/006; A01K 5/0114; A01K 5/02; G01G 17/08; G01G 21/22; G16H 50/30; G16H 20/60; G01G 19/22

(57) ABSTRACT

There are provided products, methods and systems related to identifying and fulfilling dynamic health and nutritional needs of an animal by building a predictive health and nutrition model capable of generating nutrition profiles for the animals based on collected data. The nutrition profile can be translated into a custom meal and made available to the animal for consumption.

### CROSS-REFERENCE TO RELATED APPLICATIONS

This application claims benefit to U.S. Provisional Patent Application No. 62/436,445, filed Dec. 20, 2016, which is incorporated herein by reference in its entirety in the present disclosure.

### BACKGROUND

Ensuring the optimal health and well-being of an animal can be a non-trivial endeavor requiring constant, consistent, and concerted effort, knowledge, and resources. Typically animals' welfare, such as domesticated animals and livestock, is of critical importance to their owners. There is an immediate need for resources to manage the health and quality of life of the animals by their owners.

### SUMMARY

Provided herein are unique products, methods, and systems for identifying and fulfilling nutritional needs and health of animals. The invention is related to a predictive health and nutrition model capable of generating nutrition profiles for the animals based on collected data. The nutrition profile can be translated into a custom meal product and made available to the user for consumption.

In one aspect, there are provided methods for managing dynamic health and nutritional needs of an animal, comprising: obtaining observed data of one or more dynamic characterization variables of an animal; determining derived data of the one or more dynamic characterization variables of the animal; building a dynamic predictive health model for the animal based on the observed data and the derived data; interpreting a dynamic nutrition profile of the animal based on the dynamic predictive health model; and customizing a dynamic meal for the animal based on the dynamic nutrition profile, thereby managing the dynamic health and nutritional needs of the animal.

In some embodiments of the aforementioned aspect, the obtaining, the determining, the building, the interpreting, and the customizing steps are in real-time.

In some embodiments of the aforementioned aspect and embodiments, the animal is a pet and/or livestock. In some embodiments of the aforementioned aspect and embodiments, the pet is a canine, feline, reptile, bird, rabbit, guinea pig, mice, hamster, or combinations thereof; and livestock is dairy cattle, calf, pig, sheep, goat, horse, mule, buffalo, camel, chickens, llama, yak, donkey, or combinations thereof.

In some embodiments of the aforementioned aspect and embodiments, the dynamic characterization variables are selected from the group consisting of weight, food consumption, steps taken, distance, speed, age, life stage, lifestyle, breed, injury, illness, heart rate, seasonal changes, activity level, sleep, environment, location, behavior, agility, and combinations thereof.

In some embodiments of the aforementioned aspect and embodiments, the method comprises obtaining the observed data of the one or more dynamic characterization variables using one or more sensor enabled devices. In some embodiments of the aforementioned aspect and embodiments, the one or more sensor enabled devices are a wearable device, a smart scale device, a smart feeder device, a smart scale, a bodily fluid and excrement analyzer, or combinations thereof.

In some embodiments of the aforementioned aspect and embodiments, the method further comprises determining the derived data of the one or more dynamic characterization variables from research articles, journals, professional opinion, feedback, diagnosis, health history, quality of life attributes, analysis from animal's owner, analysis of the nearest neighbor (NN), medication and medical treatment history, blood panel, DNA analysis, fecal analysis, trends therein, or combinations thereof.

In some embodiments of the aforementioned aspect and embodiments, the method further comprises determining the derived data of the one or more dynamic characterization variables of the animal from dynamic data feedback loop that constantly adds, removes, adjusts, and/or evolves the data based on the observed and the derived data of the one or more dynamic characterization variables in real-time.

In some embodiments of the aforementioned aspect and embodiments, the method further comprises determining the derived data of the one or more dynamic characterization variables of the animal from empirical data of one or more dynamic characterization variables from nearest neighbor (NN). In some embodiments, the empirical data comprises observed and derived data of the one or more dynamic characterization variables from NN.

In some embodiments of the aforementioned aspect and embodiments, the dynamic predictive health model for the animal predicts the animal's dynamic health and health growth requirements. In some embodiments of the aforementioned aspect and embodiments, the dynamic predictive health model constantly adjusts and/or evolves based on the observed and the derived data of the one or more dynamic characterization variables in real-time.

In some embodiments of the aforementioned aspect and embodiments, the dynamic nutrition profile comprises nutrient amount; supplement amount; medication amount; ratio of nutrients, supplements, and medications; and combinations thereof. In some embodiments of the aforementioned aspect and embodiments, the nutrient is macronutrient, micronutrient, dietary supplement, moisture, or combinations thereof. In some embodiments of the aforementioned aspect and embodiments, the dynamic nutrition profile is interpreted in real time by the dynamic predictive health model.

In some embodiments of the aforementioned aspect and embodiments, the customized dynamic meal addresses requirements of the dynamic nutrition profile and health goals for the animal. In some embodiments of the aforementioned aspect and embodiments, efficacy of the customized dynamic meal is reflected in the observed data of the one or more dynamic characterization variables of the animal which when fed back by the dynamic data feedback loop, it constantly adjusts, and/or evolves the derived data of the one or more dynamic characterization variables in real-time. In some embodiments of the aforementioned aspect and embodiments, the constantly adjusting and/or evolving derived data builds the dynamic predictive health model with more precision.

In some embodiments of the aforementioned aspect and embodiments, the method further comprises manufacturing or procuring the customized dynamic meal and delivering the customized dynamic meal to the animal's owner.

In some embodiments of the aforementioned aspect and embodiments, the one or more dynamic characterization variables of an animal comprises genetic information, bodily fluid information, and excrement information.

In one aspect, there is provided a system for managing dynamic health and nutritional needs of an animal, comprising:

one or more sensor enabled devices configured for obtaining and transmitting observed data related to one or more dynamic characterization variables of an animal;

a central data store, operably connected to the one or more sensor enabled devices, configured for
obtaining the transmitted observed data;
determining derived data of the one or more dynamic characterization variables of the animal;
building a dynamic predictive health model for the animal based on the observed data and the derived data; and
interpreting a dynamic nutrition profile of the animal based on the dynamic predictive and

a custom meal formulation subsystem, operably connected to the central data store, configured for obtaining the dynamic nutrition profile from the central data store and customizing a dynamic meal for the animal based on the dynamic nutrition profile,

thereby providing a system for managing dynamic health and nutritional needs of the animal.

In some embodiments of the aforementioned aspect, the one or more sensor enabled devices, the central data store, and the custom meal formulation subsystem operate in real time. In some embodiments of the aforementioned aspect and embodiments, the one or more sensor enabled devices are a wearable device, a smart scale device, a smart feeder device, or combinations thereof. In some embodiments of the aforementioned aspect and embodiments, the sensor enabled device comprises a dedicated data collector comprising one or more sensors, processing circuitry, storage media, and a wireless communication module. In some embodiments of the aforementioned aspect and embodiments, the one or more sensors obtain and transmit observed data related to physicality in three dimensions; thermodynamic properties; electromagnetic properties; hydrodynamic and aerodynamic properties; chemical properties; acoustic properties; optic properties; biological properties; and combinations thereof.

In some embodiments of the aforementioned aspect and embodiments, the one or more sensor enabled devices are configured to transmit the observed data to the animal owner's device selected from the group consisting of smart phone, tablet, computer, smart watch, computer-like device, and combinations thereof.

In some embodiments of the aforementioned aspect and embodiments, the system further comprises a base station or peer (BSoP) device operably connected to the one or more sensor enabled devices and the central data store and configured to store, compute, and/or transmit the data from the one or more sensor enabled devices to the central data store.

In some embodiments of the aforementioned aspect and embodiments, the one or more sensor enabled devices are configured to create a device-to-device (D2D) network to transmit the observed data to the BSoP and/or the central data store.

In some embodiments of the aforementioned aspect and embodiments, the D2D network comprises cognitive self-organizing/self-optimizing device-to-device (cSOSO-D2D) network; a direct device-to-device (D2D) network; and/or interconnection with other devices. In some embodiments of the aforementioned aspect and embodiments, the cSOSO-D2D network is configured to self-organize/self-optimize its interconnections with other devices.

In some embodiments of the aforementioned aspect and embodiments, the BSoP device is one or more cSOSO-D2D nodes operably connected to the one or more sensor enabled devices and the central data store and configured to participate in the cSOSO-D2D network and store and/or transmit the data from the one or more sensor enabled devices to the central data store. In some embodiments of the aforementioned aspect and embodiments, the cSOSO-D2D node is interconnected to other one or more cSOSO-D2D nodes through the cSOSO-D2D network to store and/or relay data amongst the cSOSO-D2D nodes.

In some embodiments of the aforementioned aspect and embodiments, the BSoP is configured to obtain observed data and derived data for comparing the derived data of the one or more dynamic characterization variables of the animal to its nearest neighbor (NN) and scientific literature within the CDS.

In some embodiments of the aforementioned aspect and embodiments, the central data store is configured to determine the derived data of the one or more dynamic characterization variables from research articles, journals, professional opinion, feedback, diagnosis, health history, quality of life attributes, analysis from animal's owner, analysis of the nearest neighbor (NN), or combinations thereof.

In some embodiments of the aforementioned aspect and embodiments, the central data store is configured to determine the derived data of the one or more dynamic characterization variables of the animal from dynamic data feedback loop that constantly adds, removes, adjusts, and/or evolves the data based on the observed and the derived data of the one or more dynamic characterization variables in real-time.

In some embodiments of the aforementioned aspect and embodiments, the custom meal formulation subsystem is configured to customize the dynamic meal based on health goals and meal ingredients.

In some embodiments of the aforementioned aspect and embodiments, the wearable device is configured to be worn on any body part of the animal.

In some embodiments of the aforementioned aspect and embodiments, the smart scale device is configured to obtain, track, log, and transmit observed data related to weight of the animal. In some embodiments of the aforementioned aspect and embodiments, the smart scale device comprises a smart scale and a control housing unit. In some embodiments of the aforementioned aspect and embodiments, the smart scale comprises one or more sensors and an incompressible fluid pad. In some embodiments of the aforementioned aspect and embodiments, the one or more sensors in the smart scale are configured to obtain the observed data related to the weight of the animal. In some embodiments of the aforementioned aspect and embodiments, the control housing unit in the smart scale device obtains the observed data from the one or more sensors and tracks, logs, and transmits the observed data related to the weight of the animal to the CDS and/or BSoP.

In some embodiments of the aforementioned aspect and embodiments, the smart feeder device is integrated with a smart scale and is configured to obtain, track, log, and transmit observed data related to amount of food consumption by the animal. In some embodiments of the aforementioned aspect and embodiments, the smart feeder device is further configured to obtain, track, log, and transmit observed data related to frequency of the food consumption and speed of the food consumption of the animal.

In one aspect, there is provided a sensor enabled device for animals, comprising: a dedicated data collector comprising one or more sensors, processing circuitry, storage media, and a wireless communication module, wherein the sensor enabled device is a wearable device, a smart scale device, a smart feeder device, or combinations thereof. In some embodiments of the aforementioned aspect, the smart scale device further comprises sensors and an incompressible fluid pad. In some embodiments of the aforementioned aspect and embodiments, the smart feeder device is integrated with the smart scale device.

In one aspect, there is provided a computer program product encoded on non-transitory computer-readable medium, which when executed, causes one or more computers to manage dynamic health and nutritional needs of an animal, the computer program product comprising:

instructions executable to obtain observed data of one or more dynamic characterization variables of an animal;

instructions executable to determine derived data of the one or more dynamic characterization variables of the animal;

instructions executable to build a dynamic predictive health model for the animal based on the observed data and the derived data;

instructions executable to interpret a dynamic nutrition profile of the animal based on the dynamic predictive health model; and

instructions executable to customize a dynamic meal for the animal based on the dynamic nutrition profile.

In some embodiments of the aforementioned aspect and embodiments, the computer program product further comprises instructions executable to determine the derived data of the one or more dynamic characterization variables of the animal from dynamic data feedback loop that constantly adds, removes, adjusts, and/or evolves the data based on the observed and the derived data of the one or more dynamic characterization variables in real-time.

### BRIEF DESCRIPTION OF THE DRAWINGS

The novel features of the invention are set forth with particularity in the appended claims. A better understanding of the features and advantages of the present invention may be obtained by reference to the following detailed description that sets forth illustrative embodiments, in which the principles of the invention are utilized, and the accompanying drawings of which:

FIG. 1 is an illustration of some embodiments related to the methods provided herein.

FIG. 2 is an illustration of some embodiments related to the systems provided herein.

FIG. 3 is an illustration of some embodiments related to the devices, methods, and systems provided herein.

FIG. 4 is an illustration of some embodiments related to the devices, methods, and systems provided herein.

FIG. 5 is an illustration of some embodiments related to the wearable device provided herein.

FIG. 6 is an illustration of some embodiments related to the wearable device provided herein.

FIG. 7 is an illustration of some embodiments related to the smart scale device provided herein.

FIG. 8 is an illustration of some embodiments related to the smart scale device provided herein.

FIG. 9 is an illustration of some embodiments related to the smart feeder device provided herein.

FIG. 10 is an illustration of some embodiments related to the smart feeder device provided herein.

FIG. 11 is an illustration of some embodiments related to the body fluid and excrement analyzer provided herein.

FIG. 12 is an illustration of some embodiments related to the interconnected systems, methods, products, and computer program products provided herein.

### DETAILED DESCRIPTION

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are described herein.

It is noted that, as used herein and in the appended claims, the singular forms “a,” “an,” and “the” include plural references unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods, Systems, and Products

Disclosed herein are unique methods, systems, and products for identifying and fulfilling nutritional needs and general health goals (HGs) of the animals.

### Methods

In one aspect, there are provided methods for managing dynamic health and nutritional needs of an animal, comprising: obtaining observed data (or observed data points or oDP) of one or more dynamic characterization variables (CVs) of an animal; determining derived data (or derived data points or dDP) of the one or more dynamic characterization variables of the animal; building a dynamic predictive health model (pHM) for the animal based on the observed data and the derived data (or observed and derived data points or odDP); interpreting a dynamic nutrition profile (NP) of the animal based on the dynamic predictive health model; and customizing a dynamic meal for the animal based on the dynamic nutrition profile, thereby managing the dynamic health and nutritional needs of the animal.

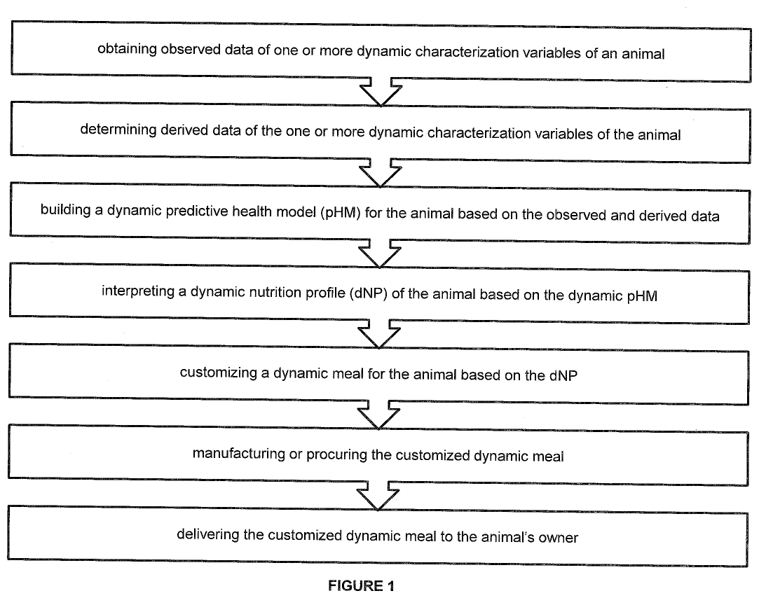

An exemplary illustration of the methods is shown in FIG. 1. As illustrated in FIG. 1, the methods provided herein relate to managing dynamic health and nutritional needs of an animal through several steps. The management of the dynamic health and nutritional needs of an animal may be carried out in real time or over a period of hours, days, months, or years. In some embodiments of the aforementioned aspect, the obtaining, the determining, the building, the interpreting, and/or the customizing steps are in real-time.

The methods comprise obtaining observed data of one or more dynamic characterization variables of an animal. The obtaining of the observed data may include one or more of collection, assimilation, sanitation, and/or transformation of observed data of the one or more dynamic characterization variables of the animal. The observed data may be quantitative and include a number, name, grade, and/or a level.

The “animal” herein includes one or more animals capable of being observed for health and nutritional needs. In some embodiments, the animal is a pet and/or livestock (PAL). In some embodiments, the animal is an animal in a zoo, national wild life park, or any other such facility. In some embodiments, the animal includes, but not limited to, canine, feline, reptile, bird, rabbit, guinea pig, mice, hamster, dairy cattle, calf, pig, sheep, goat, horse, mule, buffalo, camel, chickens, llama, yak, donkey, or combinations thereof. For example, in some embodiments, the pet is a canine, feline, reptile, bird, rabbit, guinea pig, mice, hamster, or combinations thereof; and livestock is dairy cattle, calf, pig, sheep, goat, horse, mule, buffalo, camel, chickens, llama, yak, donkey, or combinations thereof. While some examples of the animals have been described herein, any animal capable of being observed for health and nutritional needs is well within the scope of the invention.

The dynamic characterization variables of the animal can be any variable that can be related to the health and nutritional needs of the animal. The characterization variables are dynamic as they may change over a period of time. In some embodiments, the dynamic characterization variables of the animal include, but not limited to, weight, food consumption, steps taken, distance, speed, age, life stage, lifestyle, breed, injury, illness, heart rate, seasonal changes, activity level, sleep, environment, location, behavior, agility, or combinations thereof. In some embodiments, the dynamic characterization variables of the animal include, but not limited to, weight, activity level, sleep, steps taken, distance traveled, speed, heart rate, or combinations thereof. In some embodiments, the dynamic characterization variables of the animal include, but not limited to, weight, activity level, sleep, or combinations thereof. In some embodiments, the dynamic characterization variable such as the activity level may include steps taken, distance traveled, speed, heart rate, or the like. In some embodiments of the aforementioned aspect and embodiments, the one or more dynamic characterization variables of an animal comprises genetic information, bodily fluid information, excrement information, or combinations thereof. While some examples of the dynamic characterization variables have been described herein, any other dynamic characterization variable that can be related to the health and nutritional needs of the animal is well within the scope of the invention.

In some embodiments, the step of obtaining the observed data of the one or more dynamic characterization variables of the animal obviates subjective and static identification of pertinent dynamic characterization variables of the animal. For example, the step of obtaining the observed data of the one or more dynamic characterization variables of the animal may obviate one or more of subjective and non-expert interpretations of observations by the animal owner or care taker; inaccuracies and errors in manual observation; inaccuracies from assumption that all animals within a certain age range and life stage behave in a similar fashion, are of a similar health, and therefore have similar nutritional needs; questionable characterization based on breed for mixed breeds; qualitative assessment of lifestyle, etc.

In some embodiments of the aforementioned aspect and embodiments, the method comprises obtaining the observed data of the one or more dynamic characterization variables using one or more sensor enabled devices. In some embodiments of the aforementioned aspect and embodiments, the sensor enabled device is a wearable device, a smart scale device, a smart feeder device, a smart scale, or combinations thereof. The devices have been described further herein below.

In some embodiments of the aforementioned aspect and embodiments, the method further comprises transmitting the observed data of the one or more dynamic characterization variables from one sensor enabled device to another sensor enabled device (peer devices or nearest neighbor (NN) explained further herein below) before the step of obtaining the observed data of the one or more dynamic characterization variables. For example only, the observed data related to the weight of the animal may be transmitted from the smart scale device to the smart feeder device and/or the wearable device or vice versa before the observed data related to the dynamic characterization variables selected from weight (from the smart scale device); food intake (from the smart feeder device); and distance walked/ran (from the wearable collar), is obtained. For further example only, the observed data related to the activity level of one animal may be transmitted from a wearable collar of the animal to a wearable collar of another animal within the same household (peer as described herein below) before obtaining the observed data of the activity level of all the animals in the household. For yet another example only, the observed data related to the activity level of one animal may be transmitted from a wearable collar of the animal to a wearable collar of another animal within the neighborhood (NN as described herein below) before obtaining the observed data of the activity level of all the animals in the household. All of the examples described above relate to the aforementioned aspect and embodiments, wherein the method further comprises transmitting the observed data of the one or more dynamic characterization variables from one sensor enabled device to another sensor enabled device.

As illustrated in FIG. 1, the methods provided herein also comprise determining derived data (or derived data point(s)) of the one or more dynamic characterization variables of the animal. The determination of the derived data may include one or more of collection, assimilation, sanitation, mining, and/or transformation of derived data of the one or more dynamic characterization variables of the animal. The derived data may be quantitative and include a number, name, grade, and/or a level. In some embodiments, the determination of the derived data is through computation.

In some embodiments, the derived data of the one or more dynamic characterization variables is determined from research articles, journals, professional opinion, feedback, diagnosis, health history, quality of life attributes, analysis from animal's owner, analysis of the nearest neighbor (NN), medication and medical treatment history, blood panel, DNA analysis, fecal analysis, trends therein, or combinations thereof.

Although research regarding the animals and their CVs have been conducted and catalogued, it may be a daunting endeavor to extract actionable items from research articles. The burden of identifying needs and deficiencies, and how to address them properly may be a fairly challenging practice. The data may exist addressing some of the health issues and needs of the animals, however, the accessibility of this information may be very limited and typically only available to those who practice veterinary medicine, such as veterinary physicians, para-veterinary workers, and allied professionals, etc. The burden may then fall on the animal owner to locate, interpret, and implement their findings. However, this practice and treatment of ailments may be error prone and can result in serious health consequences. The computational step of determining derived data of the one or more dynamic characterization variables of the animal obviates the need for location, interpretation, and/or implementation of the available information by the animal owner.

In some embodiments, the method further comprises determining the derived data of the one or more dynamic characterization variables of the animal from dynamic data feedback loop (dDFL) that constantly adds, removes, adjusts, and/or evolves the data based on the observed and the derived data of the one or more dynamic characterization variables in real-time. For example, in some embodiments, the derived data may be weight of the pet from previous week or month such that after obtaining the observed data related to the weight of the pet, the observed data of the weight may be compared to the derived data of the weight in real-time for further analysis. Therefore, the dynamic data feedback loop may constantly add, remove, adjust, and/or evolve the data based on the observed and the derived data of the one or more dynamic characterization variables in real-time. In some embodiments, the dynamic CVs, such as seasonal changes, changes to and within the animal's environment, changes to animal's health assumptions (e.g. injury, surgery, illness, etc.) and HGs play a role in the animal's nutritional needs, and are continuously monitored and incorporated into the pHM by the dDFL for their predictive and modeling value.

In some embodiments, the methods provided herein further comprise determining the derived data of the one or more dynamic characterization variables of the animal from empirical data of one or more dynamic characterization variables from nearest neighbor (NN). In some embodiments, the empirical data comprises observed and derived data of the one or more dynamic characterization variables from the NN. The “nearest neighbor” or “NN” used herein includes other animals with similar CVs (e.g. breed, age, life stage, activity, etc.). For example, the CDS may take the CVs for Animal A and find other animals in the system such as the NN (indifferent of their physical location) with overlapping similarities (for example only, age, species, sleeping pattern, etc).

For example, the subject animal, Animal A, may have correlation values established with other animals (Animal B, C, D, etc.) in determining nearest neighbors and impact they may have on animal A's dynamic nutrition profile. The NN may be a weighted relationship of CVs correlation and may be assigned relevancy coefficients accordingly. The NN can be CV specific or may be comprised of a combination of CVs, or may correlate to the subject animal as a whole. The empirical data of the one or more dynamic characterization variables from the NN may create a crowd-sourced environment for creating and storing evidence and empirically based data of animals and animals'-NN to enable the real-time modeling and prediction of actual needs based on the observations, the interactions, and the dynamic characterization variables.

The empirical data on the trends and patterns of the one or more dynamic characterization variables from the nearest neighbor can be used in determining the derived data of the one or more dynamic characterization variables of the animal on a minute-to-minute, hour-to-hour, day-to-day, month-to-month, and/or year-to-year basis, providing an unparalleled level of insight into the animal's health.

As illustrated in FIG. 1, the methods provided herein further comprise building a dynamic predictive health model (pHM) for the animal based on the observed data and the derived data, collectively called the observed and derived data or observed and derived data points (odDP). In some embodiments, the dynamic predictive health model for the animal predicts the animal's dynamic health and health growth requirements. In some embodiments, the dynamic predictive health model constantly adjusts and/or evolves based on the observed and the derived data of the one or more dynamic characterization variables in real-time. The dynamic predictive health model built by the observed and derived data points, describes the animals' health, lifestyle, HG requirements, and other CVs as described above. The dynamic predictive health model continuously adjusts and evolves in real-time based on dynamics within the odDP optionally via the dDFL to provide greater precision, accuracy, and descriptive capabilities compared to conventional practices. The derived data may be the animal's own derived data or derived from available literature or its NN or combinations thereof, as described above.

As illustrated in FIG. 1, the methods provided herein further comprise interpreting the dynamic nutrition profile (NP) of the animal based on or by the dynamic predictive health model. In some embodiments of the aforementioned aspect and embodiments, the dynamic nutrition profile is interpreted in real time by the dynamic predictive health model. For example, typically, nutrition profile (NP) for pets and livestocks (PAL) are identified in three broad categories: puppy or kitten, adult, and senior nutrition profiles. Given the limited spectrum of differentiation between meal plans and nutrition profiles, the options available are generic and loosely based on empirical research. It is then the burden of the animal owner to determine the nutrition profile that most closely represents their animal and select the most appropriate meal plan. However, the methods provided herein provide a personalized and specific approach toward the interpretation of the dynamic nutrition profile for each animal where the NP changes over time dependent on several dynamic CVs as described above. The dynamic nature of the animals' CVs necessitates a similarly dynamic nutrition profile to ensure the continuous fulfillment of the animals' nutrition and health needs and for the attainment of health goals.

In some embodiments of the aforementioned aspect and embodiments, the dynamic nutrition profile comprises nutrient amount; supplement amount; medication amount; ratio of nutrients, supplements, and medications; and combinations thereof. In some embodiments of the aforementioned aspect and embodiments, the nutrient is macronutrient, micronutrient, dietary supplement, moisture, or combinations thereof. The macronutrients, micronutrients, and dietary supplements are well known in the art. The macronutrients include, but not limited to, fats, carbohydrates, fiber, and proteins. The micronutrients include, but not limited to, additives, vitamins, and minerals. The dietary supplements include, but not limited to, herbals, botanicals, amino acids, enzymes, nutraceuticals, and the like.

The interpretation of the dynamic nutrition profile specifies the macronutrient, micronutrient, supplementation, and/or medication amounts (may be collectively called NSM (nutrients/supplements/medicines)), their ratios, concentrations, bioavailability, and variations thereof over time. This may ensure the attainment of the HGs on a continuous basis for the animal. This is in contrast to static NPs which may presume static nutrition needs based on a mere subset of CVs that do not change over time with relation to the animal. Further, the efficacy and impact on overall health, well-being, and progress toward or away from the health goal of an implemented modification to the dynamic NP, is captured by the dDFL and fed to the pHM for incorporation. This feedback into the pHM modifies internal variables used to represent and predict the dynamic CVs such that the relationship, interdependencies, and disparities between the dynamic CVs within the pHM are modified to more closely model actual values from the animal.

As illustrated in FIG. 1, the methods provided herein further comprise customizing a dynamic meal (may be called custom PAL meal or cPM specific for PAL (pets and livestock)) for the animal based on the dynamic nutrition profile. In some embodiments of the aforementioned aspect and embodiments, the customized dynamic meal addresses requirements of the dynamic nutrition profile and health goals for the animal.

Typically, in an event an animal owner does wish to meet the nutritional needs of their animal, a combination of meals, snacks, and supplements need to be individually acquired and a significant amount of trial and error may occur in customizing a meal plan for that animal. With a limited understanding of the animal's nutritional needs and a generalized categorization of animal types, identifying the ideal meal for the animal can be extremely difficult. It may be the burden of the animal owner to prepare custom meals based on the gathered information about their animal, or to augment existing formulations with the necessary nutrients. The advantages of better result, higher accuracy, and efficient execution using the methods and systems of the invention, can obviate the ardent task of addressing the animal's nutritional needs by the animal owner.

In some embodiments of the aforementioned aspect and embodiments, efficacy of the customized dynamic meal is reflected or observed (after consumption by the animal) in the observed data of the one or more dynamic characterization variables of the animal which when fed back by the dynamic data feedback loop, it constantly adjusts, and/or evolves the derived data of the one or more dynamic characterization variables in real-time. In some embodiments of the aforementioned aspect and embodiments, the constantly adjusting and/or evolving derived data builds the dynamic predictive health model with more precision. For example, after the consumption of the customized dynamic meal by the animal, the weight of the animal may be monitored over a period of time using the smart scale device described herein. The observed data related to the weight when fed back by the dynamic data feedback loop, may adjust the derived data of the weight in real-time. This in turn may result in modifying the pHM thereby affecting the interpretation of the dynamic NP and customized meal plan.

In some embodiments of the aforementioned aspect and embodiments, the methods further comprise manufacturing or procuring the customized dynamic meal (may be called custom PAL meal or cPM specifically for PAL). The methods further comprise delivering the customized dynamic meal to the animal's owner.

Typically, the meal selection for the animals may be limited to predefined formulations and not able to address unique needs of the animal. Variations between commercially available formulations and the arbitrary selection of ingredients (e.g. salmon, chicken, beef, etc.) coupled with issues related to disparities between marketing claims and actual formulations; all may lead to significant errors.

In some embodiments of the aforementioned aspect and embodiments, the manufacturing or procuring of the customized dynamic meal is based on a custom meal formulation (cMF; or may also be called custom PAL meal formulation or cPMF for PAL). The custom meal formulation for the animals' customized dynamic meal optimizes the relationship between the following, but not limited to, customized dynamic meal goals and the customized dynamic meal ingredients. The custom meal formulation may maximize one or more of: the nutrient density such that the amount of NSM related to the dynamic NP is maximized; the completeness and coverage of all goals and ingredients over the NSMs; the utilization of ingredients that are in-season, locally available, organic, all-natural, and/or complete; the bioavailability and synergism of the goals, the ingredients, and the customized dynamic meal as a whole; the likelihood to achieve all goals with the formulation, or with minor modifications to future formulations; the flexibility and robustness of the formulation such that modifications and substitutions do not drastically unbalance or alter the underlying NSMs and health goals attainment; and the selection and use of ingredients based on recommendations and preferences of the animal owner, veterinary physician, and/or those system-generated.

The custom meal formulation may also minimize one or more of: the number of formulation iterations required to achieve maximal dynamic NP and NSM coverage; the overall costs of the ingredients, their utilization, and thereby the overall cost of the customized dynamic meal; the likelihood of unforeseen, unintended, or undesirable ingredients' effects, including but not limited to allergic reactions, sensitivities, and gastrointestinal distress; the need for significant variation of both ingredients and NSM at each formulation iteration; the likelihood of unforeseen, unintended, or undesirable ingredients and NSM interactions; and the time required to achieve health goals while ensuring optimal animals' CVs.

In some embodiments, the customized dynamic meal is manufactured in a continuous, individual, semi-batch, or batch-wise manner on a predetermined interval with the formulation as described above, generated by the systems provided herein. The customized dynamic meal produced is comprised of a dry, semi-dry, semi-moist, or moist food, or any combination thereof, using processing techniques such as, but not limited to, baking, extrusion, pressing, 3D printing, molding, spray drying, rotary drying, fluidized bed drying, freeze drying, dehydration, precipitation, vacuum processes, other food processing techniques common to the art and variations thereof. These techniques may allow for significant variability in processing, formulation, and end-product characteristics between the resulting meal, both in terms of iterations for the same animal and between animals. The customized dynamic meal may be made available to the animal owner for monitored administration to the animal, once production and packaging are complete.

### Systems

Provided herein are systems for carrying out the methods of the invention.

In one aspect, there is provided a system for managing dynamic health and nutritional needs of an animal, comprising: one or more sensor enabled devices configured for obtaining and transmitting observed data related to one or more dynamic characterization variables of an animal; a central data store, operably connected to the one or more sensor enabled devices, configured for
obtaining the transmitted observed data;
determining derived data of the one or more dynamic characterization variables of the animal;
building a dynamic predictive health model for the animal based on the observed data and the derived data; and
interpreting a dynamic nutrition profile of the animal based on the dynamic predictive health model; and

a custom meal formulation subsystem, operably connected to the central data store, configured for obtaining the dynamic nutrition profile from the central data store and customizing a dynamic meal for the animal based on the dynamic nutrition profile, thereby providing a system for managing dynamic health and nutritional needs of the animal.

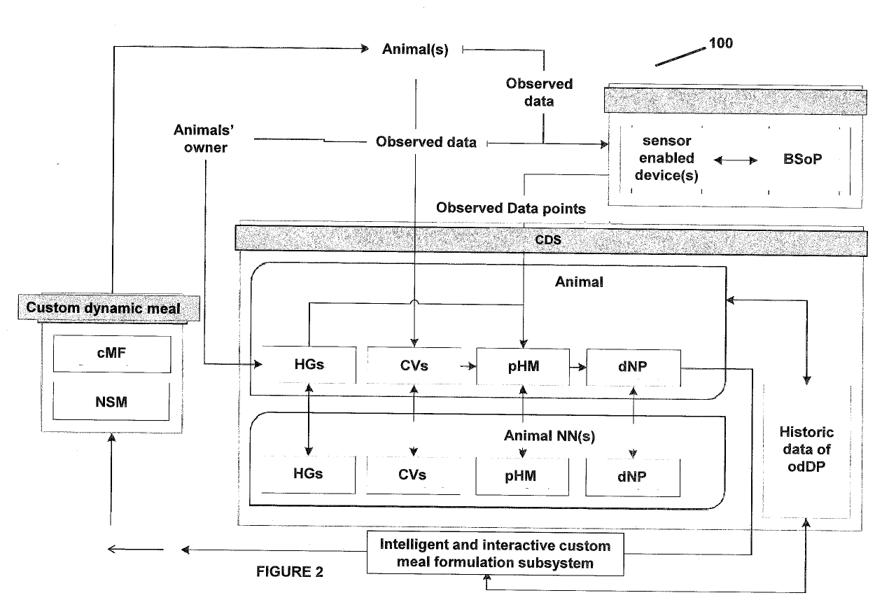

An exemplary illustration of the systems provided herein is as shown in FIG. 2. It is to be understood that all the components of the systems illustrated in FIG. 2 are not necessary to operate the system. A combination of one or more components illustrated in FIG. 2 can be used to carry out the systems of the invention. The systems provided herein facilitate integration of components into a robust hybrid system that uses sensor-enabled devices to continuously monitor the animal (such as, for example only, PAL) to collect observed and derived data, a dynamic database merging the most up-to-date information available, and the custom manufacturing of a unique animal meal capable of addressing the animal's nutritional needs on an evolving and reoccurring basis.

As illustrated in FIG. 2, the system 100 provided herein relates to managing the dynamic health and nutritional needs of an animal by using one or more sensor enabled devices configured for obtaining and transmitting the observed data related to the one or more dynamic characterization variables of the animal. In some embodiments of the aforementioned aspect, the sensor enabled devices operates in real time. It is to be understood that any conventional sensor enabled device can be integrated with the systems provided herein for managing the dynamic health and nutritional needs of an animal. In some embodiments of the aforementioned aspect and embodiments, the one or more sensor enabled devices are a wearable device, a smart scale device, a smart scale, a smart feeder device, or combinations thereof. These sensor enabled devices are described in detail further herein below.

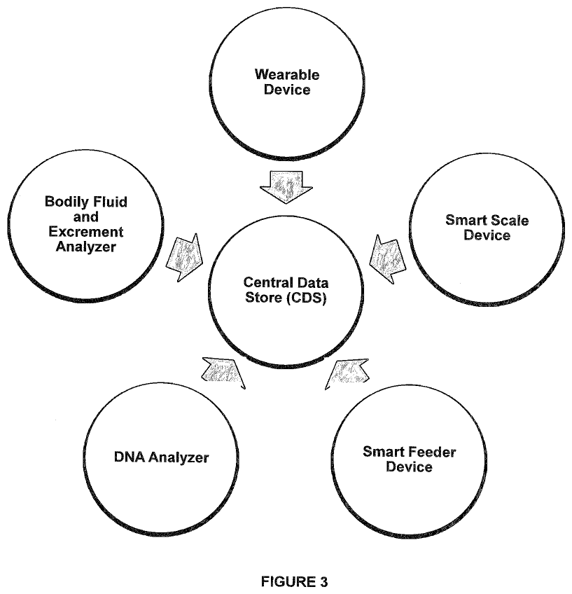

As illustrated in FIG. 2, the system 100 provided herein further comprises a central data store (CDS) operably connected to the one or more sensor enabled devices. As illustrated in FIG. 3, the central data store (CDS) may be operably connected to one or more of the sensor enabled devices selected from, but not limited to, a wearable device, a smart scale device, a smart feeder device, a bodily fluid or excrement analyzer, a DNA analyzer, etc. Observed data is constantly collected via independent devices. Each sensor enabled device is capable of transmitting the data to each other, to the base station (described herein below), and/or directly to the CDS.

The CDS is a major component of the system and evolves dynamically over time. The CDS is configured to obtain the transmitted observed data related to the one or more dynamic characterization variables of the animal from the one or more sensor enabled devices and determine the derived data of the one or more dynamic characterization variables of the animal. In some embodiments of the aforementioned aspect and embodiments, the central data store is configured to determine the derived data of the one or more dynamic characterization variables from research articles, journals, professional opinion, feedback, diagnosis, health history, quality of life attributes, analysis from animal's owner, analysis of the nearest neighbor (NN), or combinations thereof. The observed data, the derived data, and the dynamic characterization variables, have been described in detail herein above. The data may also be obtained from base station or peer (BSoP) device, described further herein below. The method of data compilation utilized combines the aggregation of both objective and derived data which are then utilized to build the dynamic predictive health model and interpret the dynamic nutrition profile (NP).

The CDS implements a unique approach whereby the disparity between the animal observations, existing knowledge found within research literature and industry, and the applicability of that knowledge to fulfill the animals' unique needs, is eliminated. Although two or more animals may be of a similar age or breed, other CVs that are unique to the animal and have significant consequences to the applicability and impact of a recommendation may have to be considered to account for the inherent variability between individual animals, as is done in this system.

In some embodiments of the aforementioned aspect and embodiments, the central data store is configured to determine the derived data of the one or more dynamic characterization variables of the animal from dynamic data feedback loop that constantly adds, removes, adjusts, and/or evolves the data based on the observed and the derived data of the one or more dynamic characterization variables in real-time. The data contained within the CDS, both observed data points and derived data points, collectively may be observed and derived data points (odDP), may be continuously growing and changing in terms of volume, variety, velocity, veracity, complexity, and predictive value. The driver for the growth and data dynamics may be the dynamic data feedback loop (dDFL as explained above), which may continuously add, remove, and adjust odDP within the CDS. The odDP may grow in scale (e.g. the total number of data points and their types) and evolve (e.g. precision, descriptive potential, and variety) at a rapid velocity, veracity (i.e. quality), and complexity as new data becomes available. New data may include additional odDP, changes to existing odDP, and adjustments to existing data assumptions which may be aggregated from the various data generators, both internal to the system and external but interfacing with the system. In some embodiments, the data may be continuously updated with new observed data points, and expanded with changes to the derived data points via feedback to provide a greater degree of accuracy to the system as a whole.

The dDFL provides a mechanism for the quantification and qualification of specific modifications to the animals' predictive health model, dynamic nutrition profile, and health goals, which may be then stored and utilized for future refinement iterations. Those refinements may be implemented by the intelligent and interactive custom meal formulation subsystem to create a custom dynamic meal by optimizing the selection of custom meal ingredients to ensure proper nutrient, supplement, and medication levels within the meal.

As illustrated in FIG. 2, the system 100 provided herein further comprises a custom meal formulation subsystem, operably connected to the central data store, and configured for obtaining the dynamic nutrition profile from the central data store and customizing a dynamic meal for the animal based on the dynamic nutrition profile. The custom meal formulation subsystem for pets and livestocks (PAL) may be called custom PAL meal formulation (cPMF) subsystem. In some embodiments of the aforementioned aspect and embodiments, the custom meal formulation subsystem is configured to customize the dynamic meal based on health goals, NSMs, and meal ingredients.

In some embodiments of the aforementioned aspect, the one or more sensor enabled devices, the central data store, and the custom meal formulation subsystem operate in real time.

In some embodiments of the aforementioned aspect and embodiments, the system 100 as illustrated in FIG. 2 further comprises a base station (BS) device or base station or peer (BSoP) device operably connected to the one or more sensor enabled devices and the central data store and configured to store, compute, and/or transmit the data from the one or more sensor enabled devices to the central data store. The “base station or peer” device or BSoP as used herein includes one or more base stations (may also be called nodes as described further herein below) communicating with each other and/or communicating with one or more sensor enabled devices. The “peer” device used herein includes any device that communicates with another such as, one sensor enabled device communicating with another and/or one base station communicating with another. The base station(s) may be more stationary/less mobile than a peer (and possess more computational and storage capacity than its peers), however, a peer may be any device that communicates with another (e.g. two base stations can communicate, or two sensor enabled devices can communicate and thus would act as peers). Further, the base station has the ability to communicate to the CDS. For example, in some embodiments, the peer device may be a wearable collar (a wearable device as described herein) transmitting to another sensor enabled device such as, but not limited to, smart scale device or smart feeder device, which then transmits to the base station and/or CDS. In some embodiments, such peer device(s) may be applicable to households with multiple animals or multiple sensor enabled devices throughout the house.

For example only, the interconnection can be done in a variety of ways with a wireless signal either directly transmitted (peer1>data>peer2>data>CDS) or indirectly routed (peer1>data>peer3>data>peer2>data>CDS). In the foregoing scenarios, all sensor enabled devices are serving as peers (communicating with each other), while peer2 may be the base station as it possesses the capacity to store the data until the CDS is available. The base station attempts to maintain a constant connection with (path to) the CDS to transmit data upon a data transmission trigger event (DTTE), explained further herein below. In some embodiments, one sensor enabled device, such as a smart feeder device may not be able to communicate with the BS and/or the CDS (bad wifi signal, low battery, etc.) and may relay the data through another smart feeder device or the wearable device to the BS and/or the CDS. Other scenarios include, but not limited to:

1. Wearable collar 1>data>smart feeder bowl 1>data>smart feeder bowl 2>data>CDS
2. Wearable collar 1>data>smart feeder bowl 1>data>CDS
3. Wearable collar 1>data>smart feeder bowl 2>data>CDS

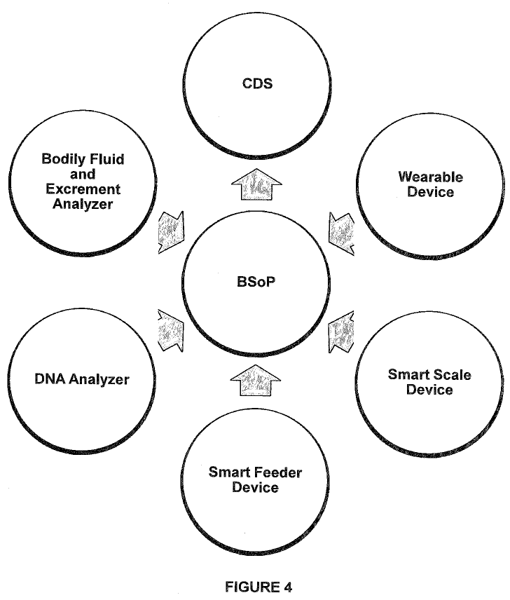

In some embodiments, the system's complexity, storage, computational power, and communication capabilities are off-loaded to BSoP. The design of the BSoP can be wall-mountable, standalone, or integrated into the design of a household item to minimize its footprint. As illustrated in FIG. 4, the BSoP may be operably connected to central data store (CDS) and one or more of the sensor enabled devices selected from, but not limited to, a wearable device, a smart scale device, a smart feeder device, a bodily fluid or excrement analyzer, a DNA analyzer, etc. The observed data is constantly collected via independent devices. In some embodiments, each device transmits data to the BSoP, at which point it is stored and transmitted to the CDS. The BSoP may allow for low energy communication methods to be implemented, reserving higher power communication to the CDS.

In some embodiments of the aforementioned aspect and embodiments, the BSoP is configured to obtain the observed data and the derived data for comparing the derived data of the one or more dynamic characterization variables of the animal to its nearest neighbor (NN) and scientific literature within the CDS. In some embodiments, the BSoP devices are capable of chronologically logging marked differences in observed data and derived data points related to the dynamic CVs providing the pHM with continuously evolving data with increased modeling and predictive precision and accuracy over time. The odDPs are then utilized, in conjunction with other observed and derived data related to the animal and optionally animal-NNs to define the animals' dynamic nutrition profile.

In some embodiments of the aforementioned aspect and embodiments, the one or more sensor enabled devices are configured to create a device-to-device (D2D) network to transmit the observed data to the BSoP and/or the central data store. In some embodiments of the aforementioned aspect and embodiments, the D2D network comprises cognitive self-organizing/self-optimizing device-to-device (cSOSO-D2D) network; a direct device-to-device (D2D) network; and/or interconnection with other devices.

In some embodiments of the aforementioned aspect and embodiments, the cSOSO-D2D network is configured to self-organize/self-optimize its interconnections with other devices. In some embodiments of the aforementioned aspect and embodiments, the BSoP device is one or more cSOSO-D2D nodes operably connected to the one or more sensor enabled devices and the central data store and configured to participate in the cSOSO-D2D network and store and/or transmit the data from the one or more sensor enabled devices to the central data store. In some embodiments of the aforementioned aspect and embodiments, the cSOSO-D2D node is interconnected to other one or more cSOSO-D2D nodes through the cSOSO-D2D network to store and/or relay data amongst the cSOSO-D2D nodes.

The cSOSO-D2D network is a networking paradigm designed to self-organize it's interconnections with other network devices to establish a connection, either direct or indirect via relaying, to the CDS for data transmission, with little to no human intervention and without knowledge of nodes a priori. Further, the cSOSO-D2D network may be self-optimizing to ensure maximum throughput, reliability, performance, and redundancy, while minimizing the number of required relay hops to the CDS. Self-organization and self-optimization may occur by implementing adaptive and predictive multiple parameter cognition, whereby a variety of network characteristics, conditions, thresholds, and goals are monitored in real-time. An intelligent sub-system, which may employ an artificial intelligence algorithm and machine learning techniques, may determine the actions, configurations of the cSOSO-D2D nodes, and thereby the network.

The BSoP device or the cSOSO-D2D nodes may possess a larger memory storage capacity than the sensor enabled device. The BSoP power supply may be more powerful thus allowing for the utilization of communication technologies with a greater range and throughput, such as a wireless or cellular network communications, and more power intensive functionality, such as data processing, compilation, and aggregation, prior to transmission to the CDS. Low energy wireless communication technologies, such as Bluetooth©, infrared, and microwaves, may be employed to reduce the overall energy consumption during data transmission within the cSOSO-D2D network.

### Devices

In one aspect, there are provided sensor enabled devices for animals. The sensor enabled device can be any device capable of sensing one or more dynamic characterization variables of the animal. In some embodiments, the sensor enabled devices are selected from, but not limited to, a wearable device, a smart scale device, a smart feeder device, a bodily fluid or excrement analyzer, a DNA analyzer, etc. The observed data may be constantly collected via independent sensor enabled devices. Each sensor enabled device is capable of transmitting the data directly to the CDS and/or BSoP.

In one aspect, there is provided a wearable device configured for obtaining and transmitting observed data related to one or more dynamic characterization variables of the animal, for managing dynamic health and nutritional needs of the animal. In some embodiments, the wearable device is operably connected to the other sensor enabled devices, the CDS and/or the BSoP, where the CDS is configured for: obtaining the transmitted observed data; determining derived data of the one or more dynamic characterization variables of the animal; building a dynamic predictive health model for the animal based on the observed data and the derived data; and interpreting a dynamic nutrition profile of the animal based on the dynamic predictive health model.

In some embodiments of the above noted aspect, the wearable device is operably connected to the other sensor enabled devices as described herein. In some embodiments, the wearable device participates in the device-to-device (D2D) network (as described above) to transmit the observed data to the BSoP and/or the central data store.

The wearable device provided herein is capable of monitoring and sensing the animals' one or more dynamic characterization variables, as described above, such as, e.g. vitals, activity levels, environment, exposures, behaviors, habits, lifestyle, location (absolute and relative), overall performance, and changes thereto. The wearable device may be fashioned to be sufficiently small as to not burden the animal when worn or carried. The wearable device can be worn on any part of the body where the dynamic CVs can be monitored including, but not limited to: the ankle, leg, neck, torso, abdomen, ear, head, hands, paws, feet, tail, back, shoulder, jaw, etc. The wearable device may be configurable depending on the required size and may be fashioned to be of any color.

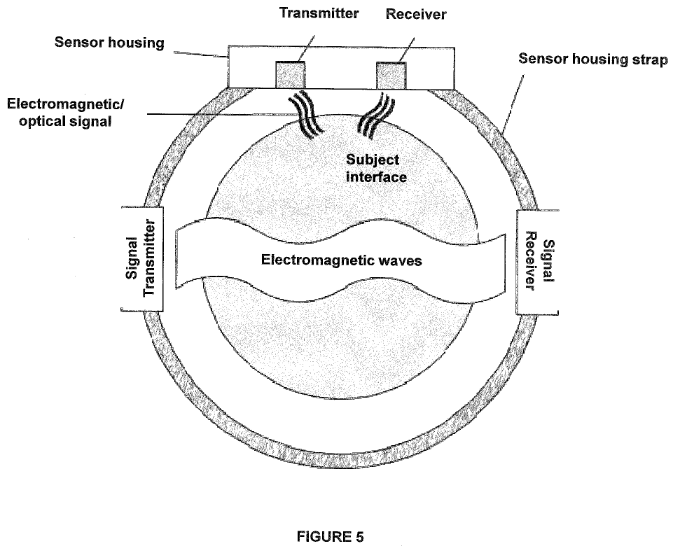

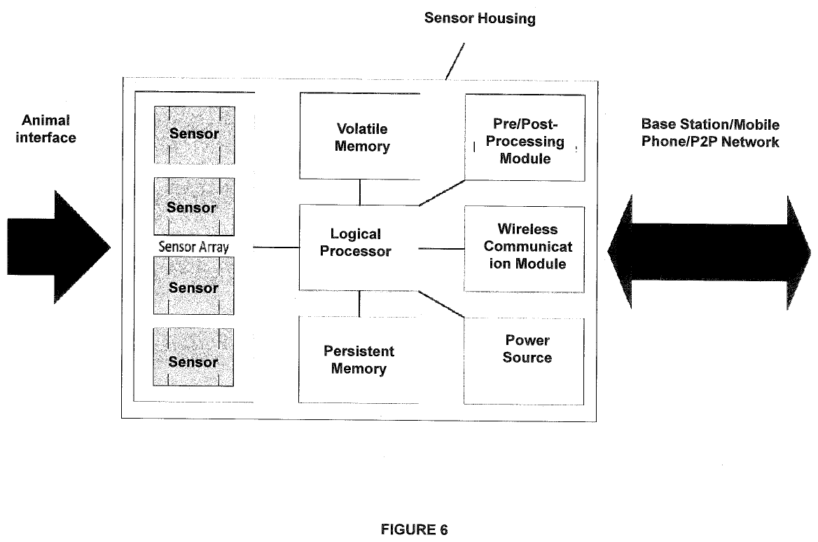

An illustration of the wearable device as a wearable collar is as shown in FIG. 5. The wearable collar as illustrated in FIG. 5 comprises a sensor housing that is configured to receive and transmit observed data related to the dynamic CVs of the animal. The wearable collar as illustrated in FIG. 5 is comprised of sensor housing suspended to a sensor housing strap which acts as the collar. The sensor housing interfaces with the animal and the animal's environment in order to gather the observed data using various transmitters and receivers. In FIG. 5, an optical transmitter and receiver are illustrated that are used for observing heart rate data where electromagnetic waves are used to detect blood circulation flow rates. A detailed illustration of the sensor housing is shown in FIG. 6 and is described further herein below.

In one aspect, there is provided a smart scale device configured for obtaining and transmitting observed data related to weight of the animal, for managing dynamic health and nutritional needs of the animal. In some embodiments, the smart scale device is operably connected to the other sensor enabled devices, the CDS and/or the BSoP, where the CDS is configured for: obtaining the transmitted observed data; determining derived data of the weight of the animal; building a dynamic predictive health model for the animal based on the observed data and the derived data of the weight; and interpreting a dynamic nutrition profile of the animal based on the dynamic predictive health model.

In some embodiments of the above noted aspect, the smart scale device is operably connected to the other sensor enabled devices as described herein. In some embodiments, the smart scale device participates in the device-to-device (D2D) network (as described above) to transmit the observed data to the BSoP and/or the CDS.

In some embodiments, the smart scale is configured for tracking and logging the weight of the animal. The smart scale device may facilitate gathering the weight information as frequently as possible with minimal interaction and subjectivity. Keeping accurate records of the animals' weight on a daily basis may contribute to a significantly more effective system. Monitoring of the weight may be a variable that plays an important role in the feedback component of the system as well and may be a key indicator of successful achievement of quantifiable and quality health goals. There is provided a unique standing and/or laying pad designed specifically for animals to gather weight information for animals of varying sizes and steadiness levels.

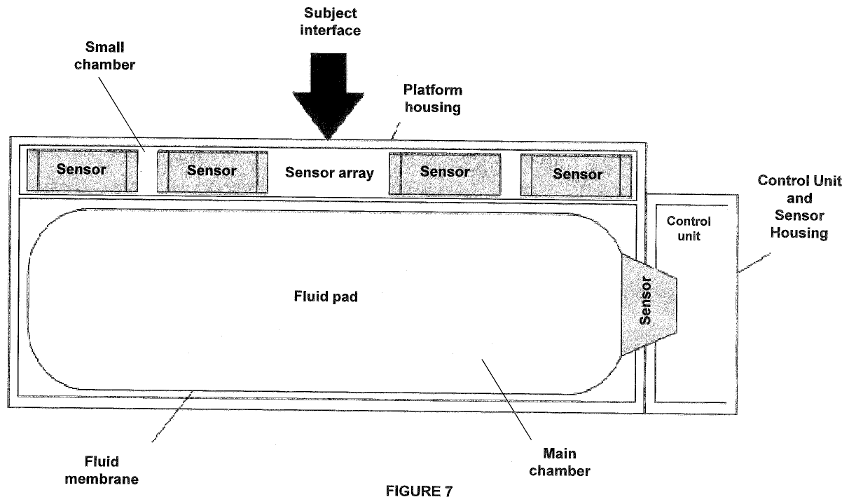

An illustration of side view of the smart scale device is as shown in FIG. 7. The smart scale device described herein comprises one or more sensors (or sensor array or array of sensors) and a fluid pad (or fluidized pad). In some embodiments, the fluid pad contains an incompressible fluid. The incompressible fluid such as any liquid may ensure that a constant volume of fluid is maintained. The fluid pad may be surrounded with a fluid membrane. The fluid membrane may be made of any appropriate leak-proof material. In some embodiments, the fluidized pad is capable of measuring weight using a non-rigid form factor. Relying on multiple connected elastic compartments, the fluid pad, and one or more displacement sensors, the weight of the animal can be determined. In some embodiments, the smart scale device captures weight inconspicuously and without force or steadiness. The measurement may take place by measuring displacement of the fluid. The amount of fluid forced out of the main chamber into a side pocket may be used to determine the force/weight of the animal. As the animal stands or lies on the pad, the incompressible fluid from the main chamber is forced into a smaller chamber lined with sensors capable of calculating the total volume of fluid displaced. The net volume (volume of fluid moved from the main chamber to the small chamber, minus the volume returned to the main chamber from the small chamber) of the fluid that is forced from the main chamber into the small chamber may be measured with a totalizer. From the net volume (based on the properties of the fluid), the open cross-sectional area of the tube connecting the main chamber and the small chamber, and correction for any flow path resistance between the chambers; the amount of force applied can be determined. From the amount of force applied, the weight of the animal applying the force can be deduced.

In some embodiments, there is provided the smart scale as described above that may not be connected to a control unit but may have a display unit that displays the weight measured by the sensors.

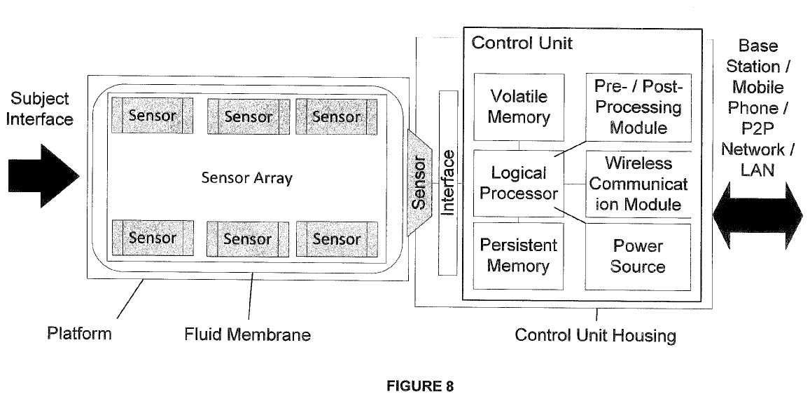

In some embodiments, the smart scale device comprising one or more sensors (or sensor array or array of sensors) and a fluid pad, further comprises a control unit housing interfacing with the one or more sensors and operably connected to the CDS and/or BSoP, configured to obtain and transmit observed data related to the weight of the animal to the other sensor enabled devices, the CDS and/or BSoP. The control unit housing is illustrated in FIG. 8 and is described further herein below.

In one aspect, there is provided a smart feeder device configured for obtaining and transmitting observed data related to the animal's eating habits, for managing dynamic health and nutritional needs of the animal. The animal's eating habit, as described herein may include one or more of amount or quantity eaten by the animal, frequency of consumption, time of consumption, etc. In some embodiments, the smart feeder device is operably connected to the other sensor enabled devices, the CDS and/or BSoP, where the CDS is configured for: obtaining the transmitted observed data of the eating habits of the animal; determining derived data of the eating habits of the animal; building a dynamic predictive health model for the animal based on the observed data and the derived data of the eating habits; and interpreting a dynamic nutrition profile of the animal based on the dynamic predictive health model.

Characterizing and quantifying the animals' eating habits may be extremely useful to the animal's owner in order to manage dynamic health and nutritional needs of the animal. This may be achieved by a custom designed smart feeder device further integrated with a weight measure or a scale for weighing the animals' meal before and after consumption.

As part of the dynamic data feedback loop (dDFL), the smart feeder device may track and log how much of the prescribed animal meal was consumed by the animal, captured by the dDFL, and fed to the pHM for incorporation. For example only, it may be anticipated that 100% of the animal meal appropriated by the system will be consumed. With this, 100% of the animal meal will need to be consumed in order for 100% of the nutritional needs to be addressed. If only 80% of the PAL meal is consumed, it can be determined that only 80% of the nutritional needs have been met. Should this become a trend for the animal, the issue may be taken into consideration and rectified accordingly by the system by building a dynamic predictive health model for the animal based on the observed data and the derived data of the eating habits; and interpreting a dynamic nutrition profile of the animal based on the dynamic predictive health model.

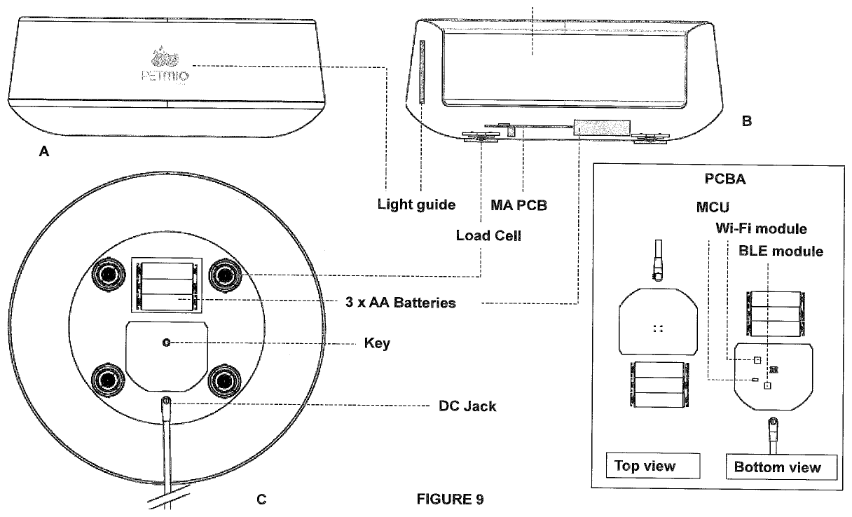

An illustration of the smart feeder device is as shown in FIG. 9. The smart feeder device illustrated in FIG. 9 is a bowl configured to measure the weight of the food consumed by the animal. A cross-sectional view of the bowl A in FIG. 9 is shown in B in FIG. 9. A bottom view of the bowl A is shown in C in FIG. 9. As illustrated, the bowl A comprises a light display, a load cell at the bottom of the bowl and MA PCB (printed circuit board). The bowl also comprises a slot for AA batteries as well as a DC jack. The load cell may be a transducer that is used to create an electrical signal whose magnitude is directly proportional to the force/weight being measured. The various load cell types include, but not limited to, hydraulic, pneumatic, and strain gauge. The smart feeder device as a bowl is designed to track the weight of the food in the bowl over time. The bowl has an inner bowl that rests on a load cell for determining the weight of the inner bowls contents (weighs the food). The weight data is saved to the bowls internal memory. The weight data is transferred from the internal memory to the CDS using an onboard Wi-Fi module. The bowl may also have a built in Bluetooth™ module for collecting data from other peers in the network such as the smart collar and the smart scale.

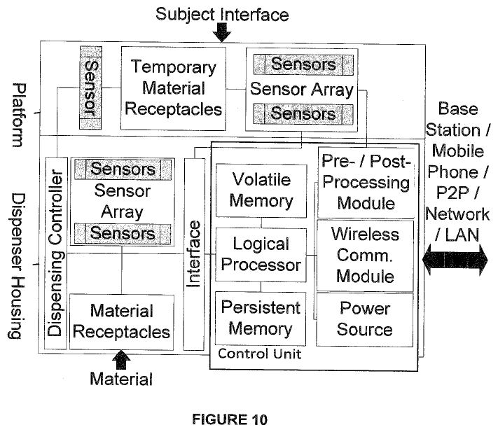

FIG. 10 illustrates a cross-sectional illustration of another smart feeder device. In some embodiments, the smart feeder device comprises one or more sensors (or sensor array or array of sensors), and a control unit housing interfacing with the one or more sensors and operably connected to the CDS and/or BSoP, configured to obtain and transmit observed data related to the eating habits of the animal to the CDS and/or BSoP. The smart feeder may be made up of platform, material receptacles, and dispensing device. The materials (food) are stored in a receptacle in a dedicated, refillable chamber. A dispensing controller may be then triggered to transfer materials from the material receptacles to the temporary material receptacles. The function of the material receptacles may be to store the food, the function of the temporary materials receptacles may be to make the food accessible to the animal during feeding. The smart feeder dispensing controller may be controlled by the control unit which may interface with the base station for dispensing instructions such as time of feeding, quantity, etc. The platform of the smart feeder device may also act as a scale using an array of weight sensors allowing for the amount dispensed from the material receptacle to the temporary material receptacle to be measured before, during, and after the feeding. The control unit may communicate with the base station using a wireless communication module. The control unit housing may be described in detail further herein below.

In one aspect, there is provided a bodily fluid and excrement analyzer configured for obtaining and transmitting observed data related to the animal's bodily fluid and excrement, for managing dynamic health and nutritional needs of the animal. In some embodiments, the bodily fluid and excrement analyzer is operably connected to the other sensor enabled devices, the CDS and/or the BSoP, where the CDS is configured for: obtaining the transmitted observed data of the bodily fluid and excrement of the animal; determining derived data of the bodily fluid and excrement of the animal; building a dynamic predictive health model for the animal based on the observed data and the derived data of the bodily fluid and excrement; and interpreting a dynamic nutrition profile of the animal based on the dynamic predictive health model.

In some embodiments, a customized probe for analyzing the composition of the animal's bodily fluid and excrement is provided. The bodily fluid and excrement analyzer is configured for determining the composition and relative quantities of the major components that make up animals' bodily fluid and excrement. The information may be utilized when examining details specific to digestion, digestibility, and absorption of NSMs. An example of the analysis by the bodily fluid and excrement analyzer is the determination of nitrogen levels. From the nitrogen levels, protein digestion can be deduced and recorded as the dynamic CV. As part of the dDFL component of the system, analyzing the composition of the excrement may allow for the quality and efficacy of the dynamic meal to be captured by the dDFL and fed to the dynamic pHM for incorporation. Modifications can then be made to the formulation to adjust NSM and ingredient quantities, ratios, and concentrations to enhance the dynamic meal to the animal.

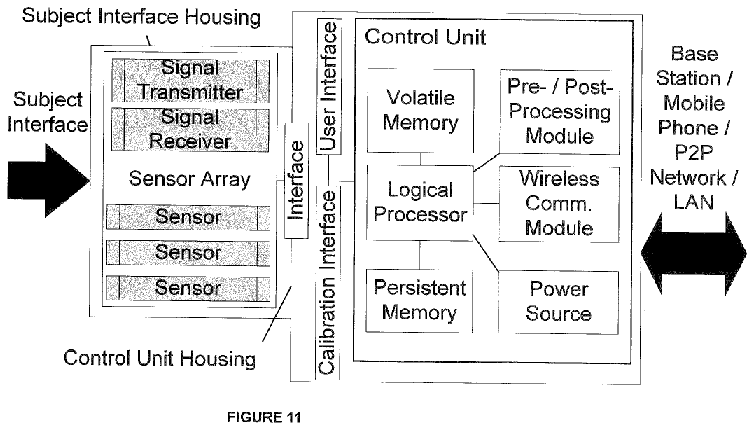

In some embodiments, the bodily fluid and excrement analyzer comprises one or more sensors (or sensor array or array of sensors), and a control unit housing interfacing with the one or more sensors and operably connected to the other sensor enabled devices, the CDS and/or BSoP, configured to obtain and transmit observed data related to the bodily fluid and excrement of the animal to the CDS and/or BSoP. The bodily fluid and excrement analyzer and its components are as illustrated in FIG. 11 and are described further herein below. The bodily fluid and excrement analyzer is comprised of sensors for capturing data from the samples, and a signal transmitter and receiver combination for capturing sample specific data. Once the data is captured it may be stored and wirelessly transmitted to the base station and/or CDS. The analyzer may have a user interface for communicating testing protocols.

In some embodiments of the aforementioned aspect and embodiments, the one or more sensors (or sensor array or array of sensors) in the sensor enabled devices described above, obtain and transmit observed data related to physicality of the animal in three dimensions; thermodynamic properties; electromagnetic properties; hydrodynamic and aerodynamic properties; chemical properties; acoustic properties; optic properties; biological properties; and combinations thereof.

The one or more sensors or sensor array may be comprised of multiple sensors that are able to identify and measure data specific to the animal and its environment. The types of sensors used and sensor readings obtained from the animal, categorized by function, include, but are not limited to, the following:

physicality in 3-dimensions: position, angle, displacement, distance, direction, velocity, acceleration, navigation, orientation, altitude, force, momentum, proximity, weight, and changes thereto;

thermodynamic properties: heat, temperature, work, energy, quantum-mechanical properties, entropy, internal/external energy, potentials, state, and changes thereto;

electromagnetic properties: electric current, electric potential, magnetic potential, ionization, polarization, wavelength, frequency, hertz, resistance, capacitative potential, and changes thereto;

hydrodynamic and aerodynamic properties: flow, fluid volume, fluid velocity, pressure, density, temperature, and changes thereto;

chemical properties: chemical, reactivity, interactivity, density, volume, flammability, stability, toxicity, material, composition, charge, molecular weight, solubility, PH, and changes thereto;

acoustic properties: frequency, wavelength, sound, vibration quantities, pressure, transduction, ranges thereof, and changes thereto;

optic properties: optical, light, imaging, photonics properties, radiation properties, diffraction, interference, wave properties and effects, reflectivity, refractions, magnification, polarization, and changes thereto; and

biological properties: vital and biological metrics, including metabolic rate and characteristics, body temperature, blood pressure, pulse rate, oxygen levels, carbon dioxide levels, and changes thereto.

The sensor housing in the wearable device (FIG. 6); the control unit housing in the smart scale device (FIG. 8); the control unit housing in the smart feeder device (FIG. 10); and the control unit housing in the bodily fluid and excrement analyzer (FIG. 11), house a dedicated data collector (dDC) that comprises an intelligent array of sensors (as described above), processing circuitry, storage media, and a wireless communication module, among other circuitry, interconnections thereof, and components. The dDC in the sensor housing of the wearable device is as illustrated in FIG. 6; the dDC in the control unit housing of the smart scale device is as illustrated in FIG. 8; the dDC in the control unit housing of the smart feeder device is as illustrated in FIG. 10; and the dDC in the control unit housing of the bodily fluid and excrement analyzer is as illustrated in FIG. 11. Depending on the desired functionality of the specific device, the intended species of the subject, the desired granularity and types of data collected, and specific sensors implemented; additional or fewer components may be incorporated.

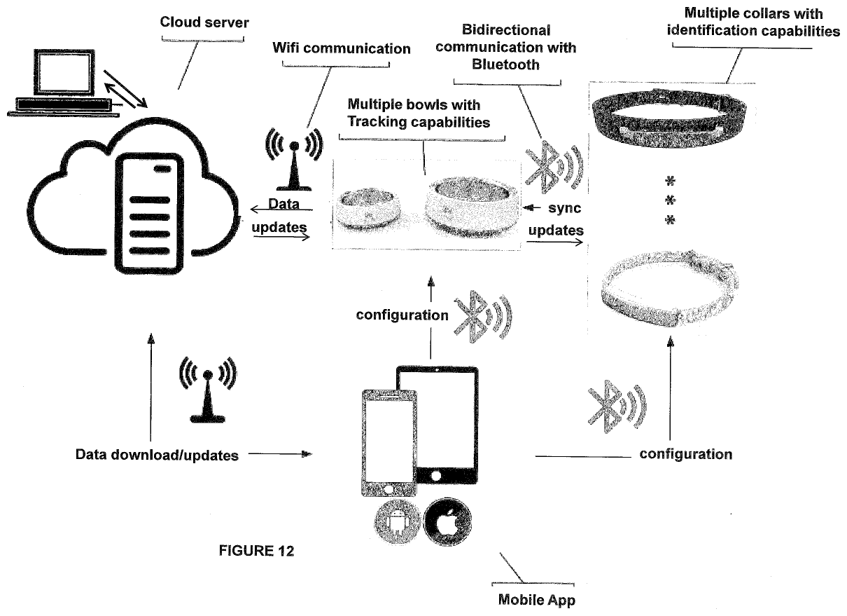

An illustration of the interconnected systems, methods, and products provided herein is as shown in FIG. 12. The sensor enabled devices, described above, are multi-functional units that can pair with the animal owner's smart phone, tablet, computer, smart watch, or computer-like or enabled device, such as a base station (BS) or other Wi-Fi connected device. The sensor data may be stored locally until an event triggers data transmission. Data transmission trigger events (DTTE) are those events in which transmission to the CDS, either via direct or indirect means, can be accomplished reliably to be stored and managed. Additionally, the sensor enabled device may create a cognitive self-organizing/self-optimizing device-to-device network, direct device-to-device network, or interconnection with other connected and associated devices, as described above, to relay data to the CDS and/or BSoP. In some embodiments, the devices are designed with sufficient volatile and persistent storage capacity to maintain data for data transfer to occur to the CDS and/or BSoP.

In some embodiments, there is also provided method for gathering DNA and genetic information specific to the animal. In some embodiments, there is provided a home kit for extracting DNA samples for use with the animal. In addition, the animal owner may authorize their veterinarian to take DNA samples and share the results. As part of the overall system, providing monthly, quarterly, or annual DNA results can be another dynamic CV that would provide data for incorporation in the predictive health model of the animal. As part of the feedback component, monitoring levels within the DNA samples can allow for an extra layer of information not otherwise achievable. The inclusion of periodic DNA sampling may provide another means of identifying similarities between animals' transcending broader comparisons such as age, breed, lifestyle, etc. and contribute to the animal-NN identification. Finding overlaying similarities between animals' DNA may increase the information available for constructing dynamic predictive health models.

In some embodiments, there is provided Smartphone application, website, and/or standalone display that allows for monitoring of the dynamic CVs transmitted between the sensor enabled devices in the overall system. Managing the system's settings and configurable options, including, but not limited to, connectivity, power levels, transmission frequency, through a Smartphone application or website may allow the animal owner a user-friendly interface for managing the overall system and its devices. Additional odDP may be gathered directly from the animal owner or veterinary physician to ascertain the perceived satisfaction, effects, acceptance, and other qualitative and quantitative data points, both of the animal and the owner. The data-gathering instrument includes, but is not limited to, surveys and/or questions posed directly to the animal owner or veterinary physician, by a person, automated system, or intelligent system, such as a conversation/messaging bot, in a manner that may be intelligent, non-intrusive, convenient, and that seamlessly integrates the data and its changes into the CDS. In addition, progressive data may be aggregated of all the information gathered, including odDP, and statistical comparison with the animal NN's. This information may be the same data used to define the dynamic nutrition profile, providing transparency to the motivating factors of modifications to the dynamic predictive health model and therefore the dynamic meal, giving confidence to the animal owner that HGs are being addressed on a continuous basis.

In some embodiments, the system described herein has multiple points of monetization, both explicitly and implicitly stated, and those methods of monetization are to be considered an embodiment of the invention. In some embodiments, the devices are provided free to animal owners who agree to a subscription plan of various lengths, monthly, quarterly, yearly, a variation or combination thereof. In some embodiments, the devices are purchased a la carte and separated from the dynamic meal. In some embodiments, the devices may be leased, or discounted, to the animal owner dependent on the dynamic meal subscription length or other qualifying event. In some embodiments of the system, all the specified devices are implemented and a subscription plan is maintained for a significant time period.

### Computer Program Product

In one aspect, there is provided a computer program product encoded on non-transitory computer-readable medium, which when executed, causes one or more computers to manage dynamic health and nutritional needs of an animal, the computer program product comprising:

instructions executable to obtain observed data of one or more dynamic characterization variables of an animal;

instructions executable to determine derived data of the one or more dynamic characterization variables of the animal;

instructions executable to build a dynamic predictive health model for the animal based on the observed data and the derived data;

instructions executable to interpret a dynamic nutrition profile of the animal based on the dynamic predictive health model; and

instructions executable to customize a dynamic meal for the animal based on the dynamic nutrition profile.

An illustration of the computer program product encoded on non-transitory computer-readable medium, which when executed, causes one or more computers to manage dynamic health and nutritional needs of an animal, is as shown in FIG. 12. The illustration of the computer in FIG. 12 is for example only. In some embodiments, the CDS is a computer and/or BSoP is a computer. In some embodiments, the computer program product executes all the instructions in real time.

In some embodiments, the computer program product further comprises instructions executable to obtain the observed data of the one or more dynamic characterization variables of the animal from a sensor enabled device. In some embodiments, the sensor enabled device is selected from, but not limited to, a wearable device, a smart scale device, a smart feeder device, or combinations thereof.

In some embodiments, the computer program product further comprises instructions executable to store, compute, and/or transmit the data from the sensor enabled device to the central data store (CDS) and/or base station or peer (BSoP).

In some embodiments, the computer program product further comprises instructions executable to create a device-to-device (D2D) network to transmit the observed data to the BSoP and/or the CDS. In some embodiments, the D2D network comprises cognitive self-organizing/self-optimizing device-to-device (cSOSO-D2D) network; a direct device-to-device (D2D) network; and/or interconnection with other devices. In some embodiments, the cSOSO-D2D network is configured to self-organize/self-optimize its interconnections with other devices.

In some embodiments, the computer program product further comprises instructions executable to determine the derived data of the one or more dynamic characterization variables of the animal in the CDS. In some embodiments, the computer program product further comprises instructions executable to determine the derived data of the one or more dynamic characterization variables from research articles, journals, professional opinion, feedback, diagnosis, health history, quality of life attributes, analysis from animal's owner, analysis of the nearest neighbor (NN), or combinations thereof. In some embodiments, the computer program product further comprises instructions executable to determine the derived data of the one or more dynamic characterization variables of the animal from dynamic data feedback loop that constantly adds, removes, adjusts, and/or evolves the data based on the observed and the derived data of the one or more dynamic characterization variables in real-time.

In some embodiments, the computer program product further comprises instructions executable to obtain the observed data and the derived data for comparing the derived data of the one or more dynamic characterization variables of the animal to its nearest neighbor (NN) and scientific literature within the CDS.

In some embodiments, the computer program product further comprises instructions executable to constantly adjust and/or evolve based on the observed and the derived data of the one or more dynamic characterization variables in real-time.

In some embodiments, the computer program product further comprises instructions executable to interpret the dynamic nutrition profile of the animal based on the dynamic predictive health model in real time.

Although the present invention, and its advantages, have been described in detail within this document, it should be understood that various changes, substitutions, and alterations can be made herein without departing from the spirit, scope, and intention of the invention as defined by the appended sentences. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function, or achieve substantially the same result as the corresponding embodiments described herein, can be utilized according to the present invention. Accordingly, the appended sentences are intended to include within their scope such processes, machines, manufacture, compositions of matter, means, methods, or steps.

For purposes of illustration, the systems, products and processes are described in the context of the animals for the users of the wearable device. As will be apparent, however, the disclosed products and processes can also be useful for other purposes or types of users. The functionality of the wearable and other sensor-enabled devices, the type of data is it able to gather, as well as the way it communicates to the central database, can all be modified to achieve the same result. The processing of the data to generate useful profiles, models, and recommendations, the use of multiple approaches, algorithms, or filtering techniques can also be modified to be a unique approach to achieving the same result. The means by which products are manufactured, acquired, delivered, or the monetizing scheme that takes effect once the nutrition profiles are generated can also be modified to be unique, while achieving the same result.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Various modifications of the invention in addition to those described herein will become apparent to those skilled in the art from the foregoing description and accompanying figures. Such modifications fall within the scope of the appended claims. Efforts have been made to ensure accuracy with respect to numbers used but some errors and deviations should be accounted for.

In the examples and elsewhere, abbreviations have the following meanings:

| | |
|---|---|
| BS = | base station |
| BSoP = | base station or peer |
| CDS = | central data store |
| cMF = | custom meal formulation |
| cPMI = | custom PAL meal goals and ingredients |
| cPMF = | custom PAL meal formulation |
| cSOSO-D2D = | cognitive self-organizing/self-optimizing device-to-device |
| CV = | characterization variable |
| dDC = | dedicated data collector |
| dD2D = | direct device-to-device |
| dDFL = | dynamic data feedback loop |
| DTTE = | data transmission trigger events |
| HGs = | health goals |
| NN = | nearest neighbor |
| NP = | nutrition profile |
| NSM = | nutrient, supplement, and medication |
| oDP = | observed data points |
| odDP = | observed and derived data points |
| PAL = | pets and livestock |
| PAL-NN = | PAL and nearest neighbor |
| pHM = | predictive health model |

### EXAMPLES

### Example 1

### Use of the Wearable Device

The wearable device is worn on the animals' body. The wearable device is a multi-functional unit that pairs with the animal owner's smart phone, tablet, computer, smart watch, or computer-like or enabled device, such as a base station or other Wi-Fi connected device. The sensor data is stored locally until an event triggers data transmission. Data Transmission Trigger Events (DTTE) are those events in which transmission to the CDS and/or the BSoP, either via direct or indirect means, can be accomplished reliably to be stored and managed. Additionally, the wearable device may create a cognitive self-organizing/self-optimizing device-to-device network, direct device-to-device network, or interconnection with other connected and associated devices to relay data to the CDS and/or the BSoP. The device is designed with sufficient volatile and persistent storage capacity to maintain data for data transfer to occur to the CDS and/or the BSoP. The BSoP continuously aggregates the data from the wearable and other devices and it transmits the data to the CDS, on a predetermined or as-needed basis.

### Example 2

### Use of the Wearable Device and NN

The wearable device gathered data from the pet. The gathered data was then transferred to the bowl using Bluetooth. The Bowl uploaded the data to the CDS using Wi-Fi. Once in the CDS, the data was interpreted and incorporated into the dynamic pHM of the pet. The CDS built the dynamic pHM by storing and processing all the information available about the pet (gathered from sensor enabled devices and pet owner's supplied data) and compared it with other pets in the system (NN) and industry knowledge that was applicable to the pet. The predictive health model then updated itself to reflect all the new data gathered and then generated a dynamic nutrition profile for the pet. The profile defined the nutritional needs of the pet in the form of macro and micro nutrients requirements (by mass). The macro and micro nutrient requirements were then translated into a food formulation identifying the specific foods necessary to achieve the required macro and micro nutrients defined by the dynamic nutrient profile. The food formulation was then sent to a fulfillment center where the food was produced, packaged, and shipped to the pet owner. The food was then consumed by the pet, and the performance and behavior of the pet was monitored with careful consideration of the impact the formulated food had on the pet.

### Example 3

### Use of the Wearable Device and NN

The wearable device gathers data from the pet. The gathered data is then transferred to the BS using Bluetooth (the Bowl in this case). The Bowl uploads the data to CDS using Wi-Fi. Once in the CDS, the data is interpreted and incorporated into the dynamic pHM of the pet. The dynamic pHM of the pet is compared with other pets in the system (Nearest Neighbors (NN)), industry knowledge that is applicable to the pet, and historic data of the pet. The dynamic pHM identifies that the majority of its nearest neighbors' are 7 lbs less in weight and the ideal size and weight from journals is 5 lbs less. The predictive health model then updates itself to reflect all the new data gathered, and generates a dynamic nutrition profile for the pet incorporating a weight loss goal. The profile defines the nutritional needs of the pet in the form of macro and micro nutrients requirements (by mass) and reduces the caloric content of the food by 2%. The macro and micro nutrient requirements are then translated into a food formulation identifying the specific foods necessary to achieve the required macro and micro nutrients defined by the dynamic nutrient profile. The food formulation is then sent to a fulfillment center where the food is produced, packaged, and shipped to the pet owner. The food is then consumed by the pet, and the performance and behavior of the pet is monitored with careful consideration of the impact the reduced calories formulated food has on the pet. The weight of the pet is then tracked for impact of calorie reduction and caloric modification is adjusted accordingly.

### Example 4

### Use of the Wearable Device, the Smart Feeder Device, and NN

The wearable device gathers data from the pet. The gathered data is then transferred to the BS using Bluetooth (the Bowl in this case). The Bowl uploads the data to CDS using Wi-Fi. Once in the CDS, the data is interpreted and incorporated into the dynamic pHM of the pet. The dynamic pHM of the pet is compared with other pets in the system (Nearest Neighbors (NN)), industry knowledge that is applicable to the pet, and historic data of the pet. The dynamic pHM identifies that the pet has expended more energy during the months of June and July in the previous two years than May or August. In preparation for the food for the month of June, the dynamic pHM anticipates that the pet will follow this trend and increases the caloric content of the food to match the average needed for the previous two months of June. The predictive health model then updates itself to reflect all the new data gathered, and generates a dynamic nutrition profile for the pet incorporating an increase in caloric content and appropriate nutritional needs for the increase in activity. The profile defines the nutritional needs of the pet in the form of macro and micro nutrients requirements (by mass) and increases the caloric content of the food by the appropriate percentage. The macro and micro nutrient requirements are then translated into a food formulation identifying the specific foods necessary to achieve the required macro and micro nutrients defined by the dynamic nutrient profile. The food formulation is then sent to a fulfillment center where the food is produced, packaged, and shipped to the pet owner. The food is then consumed by the pet, and the performance and behavior of the pet is monitored with careful consideration of the impact the increase in calories formulated food has on the pet. Using the smart feeder device, the weight of the pet is then tracked for impact of calorie increase and caloric modification is adjusted accordingly. The amount of calories expended for June are then tracked and used to gauge the consistency of the trend moving forward.

What is claimed is:

1. A method for managing dynamic health and nutritional needs of an animal, comprising:
obtaining observed data of one or more dynamic characterization variables of an animal;
determining derived data of the one or more dynamic characterization variables of the animal;
building a dynamic predictive health model for the animal based on the observed data and the derived data;
interpreting a dynamic nutrition profile of the animal based on the dynamic predictive health model; and
customizing a dynamic meal for the animal based on the dynamic nutrition profile, thereby managing the dynamic health and nutritional needs of the animal.

2. The method of claim 1, wherein the obtaining, the determining, the building, the interpreting, and the customizing steps are in real-time.

3. The method of claim 1 or 2, wherein the animal is a pet and/or livestock.

4. The method of claim 3, wherein the pet is a canine, feline, reptile, bird, rabbit, guinea pig, mice, hamster, or combinations thereof; and livestock is dairy cattle, calf, pig, sheep, goat, horse, mule, buffalo, camel, chickens, llama, yak, donkey, or combinations thereof.

5. The method of any one of the preceding claims, wherein the dynamic characterization variables are selected from the group consisting of weight, food consumption, steps taken, distance, speed, age, life stage, lifestyle, breed, injury, illness, heart rate, seasonal changes, activity level, sleep, environment, location, behavior, agility, and combinations thereof.

6. The method of any one of the preceding claims, comprising obtaining the observed data of the one or more dynamic characterization variables using one or more sensor enabled devices.

7. The method of claim 6, wherein the sensor enabled device is a wearable device, a smart scale device, a smart feeder device, a smart scale, a bodily fluid and excrement analyzer, or combinations thereof.

8. The method of any one of the preceding claims, further comprising determining the derived data of the one or more dynamic characterization variables from research articles, journals, professional opinion, feedback, diagnosis, health history, quality of life attributes, analysis from animal's owner, analysis of the nearest neighbor (NN), medication and medical treatment history, blood panel, DNA analysis, fecal analysis, trends therein, or combinations thereof.

9. The method of any one of the preceding claims, further comprising determining the derived data of the one or more dynamic characterization variables of the animal from dynamic data feedback loop that constantly adds, removes, adjusts, and/or evolves the data based on the observed and the derived data of the one or more dynamic characterization variables in real-time.

10. The method of any one of the preceding claims, further comprising determining the derived data of the one or more dynamic characterization variables of the animal from empirical data of one or more dynamic characterization variables from nearest neighbor (NN).

11. The method of claim 10, wherein the empirical data comprises observed and derived data of the one or more dynamic characterization variables from NN.

12. The method of any one of the preceding claims, wherein the dynamic predictive health model for the animal predicts the animal's dynamic health and health growth requirements.

13. The method of any one of the preceding claims, wherein the dynamic predictive health model constantly adjusts and/or evolves based on the observed and the derived data of the one or more dynamic characterization variables in real-time.

14. The method of any one of the preceding claims, wherein the dynamic nutrition profile comprises nutrient amount; supplement amount; medication amount; ratio of nutrients, supplements, and medications; and combinations thereof.

15. The method of claim 14, wherein the nutrient is macronutrient, micronutrient, dietary supplement, moisture, or combinations thereof.

16. The method of any one of the preceding claims, wherein the dynamic nutrition profile is interpreted in real time by the dynamic predictive health model.

17. The method of any one of the preceding claims, wherein the customized dynamic meal addresses requirements of the dynamic nutrition profile and health goals for the animal.

18. The method of any one of the claims 9-17, wherein efficacy of the customized dynamic meal is reflected in the observed data of the one or more dynamic characterization variables of the animal which when fed back by the dynamic data feedback loop, it constantly adjusts, and/or evolves the derived data of the one or more dynamic characterization variables in real-time.

19. The method of claim 18, wherein the constantly adjusting and/or evolving derived data builds the dynamic predictive health model with more precision.

20. The method of any one of the preceding claims, further comprising manufacturing or procuring the customized dynamic meal and delivering the customized dynamic meal to the animal's owner.

21. The method of any one of the preceding claims, wherein the one or more dynamic characterization variables of an animal comprises genetic information, bodily fluid information, excrement information, or combinations thereof.

22. A system for managing dynamic health and nutritional needs of an animal, comprising:
one or more sensor enabled devices configured for obtaining and transmitting observed data related to one or more dynamic characterization variables of an animal;
a central data store, operably connected to the one or more sensor enabled devices, configured for
obtaining the transmitted observed data;
determining derived data of the one or more dynamic characterization variables of the animal;
building a dynamic predictive health model for the animal based on the observed data and the derived data; and
interpreting a dynamic nutrition profile of the animal based on the dynamic predictive health model; and
a custom meal formulation subsystem, operably connected to the central data store, configured for obtaining the dynamic nutrition profile from the central data store and customizing a dynamic meal for the animal based on the dynamic nutrition profile,
thereby providing a system for managing dynamic health and nutritional needs of the animal.

23. The system of claim 22, wherein one or more sensor enabled devices, the central data store, and the custom meal formulation subsystem operate in real time.

24. The system of claim 22 or 23, wherein the sensor enabled device is a wearable device, a smart scale device, a smart feeder device, or combinations thereof.

25. The system of any one of the claims 22-24, wherein the sensor enabled device comprises a dedicated data collector comprising one or more sensors, processing circuitry, storage media, and a wireless communication module.

26. The system of claim 25, wherein the one or more sensors obtain and transmit observed data related to physicality in three dimensions; thermodynamic properties; electromagnetic properties; hydrodynamic and aerodynamic properties; chemical properties; acoustic properties; optic properties; biological properties; and combinations thereof.

27. The system of any one of the claims 22-26, wherein the sensor enabled device is configured to transmit the observed data to the animal owner's device selected from the group consisting of smart phone, tablet, computer, smart watch, computer-like device, and combinations thereof.

28. The system of any one of the claims 22-27, further comprising a base station or peer (BSoP) device operably connected to the one or more sensor enabled devices and the central data store and configured to store, compute, and/or transmit the data from the sensor enabled devices to the central data store.

29. The system of any one of the claims 22-28, wherein the one or more sensor enabled devices are configured to create a device-to-device (D2D) network to transmit the observed data to the BSoP and/or the central data store.

30. The system of claim 29, wherein the D2D network comprises cognitive self-organizing/self-optimizing device-to-device (cSOSO-D2D) network; a direct device-to-device (D2D) network; and/or interconnection with other devices.

31. The system of claim 30, wherein the cSOSO-D2D network is configured to self-organize/self-optimize its interconnections with other devices.

32. The system of claim 28, wherein the BSoP device is one or more cSOSO-D2D nodes operably connected to the one or more sensor enabled devices and the central data store and configured to participate in the cSOSO-D2D network and store and/or transmit the data from the one or more sensor enabled devices to the central data store.

33. The system of claim 32, wherein the cSOSO-D2D node is interconnected to other one or more cSOSO-D2D nodes through the cSOSO-D2D network to store and/or relay data amongst the cSOSO-D2D nodes.

34. The system of any one of the claims 28-33, wherein the BSoP is configured to obtain observed data and derived data for comparing the derived data of the one or more dynamic characterization variables of the animal to its nearest neighbor (NN) and scientific literature within the CDS.

35. The system of any one of the claims 22-34, wherein the central data store is configured to determine the derived data of the one or more dynamic characterization variables from research articles, journals, professional opinion, feedback, diagnosis, health history, quality of life attributes, analysis from animal's owner, analysis of the nearest neighbor (NN), or combinations thereof.

36. The system of any one of the claims 22-35, wherein the central data store is configured to determine the derived data of the one or more dynamic characterization variables of the animal from dynamic data feedback loop that constantly adds, removes, adjusts, and/or evolves the data based on the observed and the derived data of the one or more dynamic characterization variables in real-time.

37. The system of any one of the claims 22-36, wherein the custom meal formulation subsystem is configured to customize the dynamic meal based on health goals and meal ingredients.

38. The system of any one of the claims 24-37, wherein the wearable device is configured to be worn on any body part of the animal.

39. The system of any one of the claims 24-37, wherein the smart scale device is configured to obtain, track, log, and transmit observed data related to weight of the animal.

40. The system of claim 39, wherein the smart scale device comprises sensors and an incompressible fluid pad.

41. The system of any one of the claims 24-37, wherein the smart feeder device is integrated with a smart scale and is configured to obtain, track, log, and transmit observed data related to amount of food consumption by the animal.

42. The system of claim 41, wherein the smart feeder device is further configured to obtain, track, log, and transmit observed data related to frequency of the food consumption and speed of the food consumption of the animal.

43. A sensor enabled device for animals, comprising: a dedicated data collector comprising one or more sensors, processing circuitry, storage media, and a wireless communication module, wherein the sensor enabled device is a wearable device, a smart scale device, a smart feeder device, or combinations thereof.

44. The sensor enabled device of claim 43, wherein the smart scale device further comprises one or more sensors and an incompressible fluid pad.

45. The sensor enabled device of claim 43, wherein the smart feeder device is integrated with the smart scale device.

46. A computer program product encoded on non-transitory computer-readable medium, which when executed, causes one or more computers to manage dynamic health and nutritional needs of an animal, the computer program product comprising:
instructions executable to obtain observed data of one or more dynamic characterization variables of an animal;
instructions executable to determine derived data of the one or more dynamic characterization variables of the animal;
instructions executable to build a dynamic predictive health model for the animal based on the observed data and the derived data;
instructions executable to interpret a dynamic nutrition profile of the animal based on the dynamic predictive health model; and
instructions executable to customize a dynamic meal for the animal based on the dynamic nutrition profile.

47. The computer program product of claim 46, further comprising instructions executable to determine the derived data of the one or more dynamic characterization variables of the animal from dynamic data feedback loop that constantly adds, removes, adjusts, and/or evolves the data based on the observed and the derived data of the one or more dynamic characterization variables in real-time.
